(19) United States
(12) Patent Application Publication
(10) Pub. No.: US 2018/0312802 A1
(43) Pub. Date: Nov. 1, 2018

(54) METHODS FOR MODULATING PRODUCTION PROFILES OF RECOMBINANT PROTEINS IN PERFUSION MODE

(71) Applicant: ARES TRADING S.A., AUBONNE (CH)

(72) Inventors: JEAN-MARC BIELSER, GLAND (CH); MARTIN JORDAN, ECUBLENS (CH)

(21) Appl. No.: 15/772,819

(22) PCT Filed: Nov. 1, 2016
(86) PCT No.: PCT/EP2016/076304

§ 371 Date: May 2, 2018

(30) Foreign Application Priority Data
Nov. 2, 2015 (EP) 15192640.9
Jan. 12, 2016 (EP) 16150910.4

(51) Int. Cl. C12N 5/00; C07K 16/00; C12P 21/00

(52) U.S. Cl. CPC C12N 5/0018; C07K 16/00; C12N 2500/60; C12N 2510/02; C12N 2500/95; C12P 21/00

(57) ABSTRACT

The invention is in the field of cell culture. Particularly the invention relates to methods of culturing a mammalian host cell expressing a recombinant protein in perfusion mode, using a concentrated cell culture medium.

### FIELD OF THE INVENTION

The invention is in the field of cell culture. Particularly the invention relates to methods of culturing a host cell expressing a recombinant protein in perfusion mode, using a concentrated cell culture medium.

### BACKGROUND OF THE INVENTION

Optimisation of culture conditions to obtain the greatest possible productivity is one of the main aim of recombinant protein production. Even marginal increases in productivity can be significant from an economical point of view. Many commercially relevant proteins are produced recombinantly in host cells. This leads to a need to produce these proteins in an efficient and cost effective manner. Unfortunately, one of the drawback of recombinant protein production is that the conditions in which cell culture is performed usually favors a reduction of cell viability over time, reducing both efficiency and overall productivity.

Perfusion culture, batch culture and fed batch culture are the basic methods for culturing animal cells for producing recombinant proteins. Cell cultures using perfusion have been used for decades in the biotechnology industry. Indeed, perfusion mode is an effective mean to reach high yields of proteins produced recombinantly. This mode of culture has also the advantages that lower size bioreactors can be used to obtain at least similar yields compared to fed-batch cultures for instance.

Very often, especially in perfusion methods, inducing agents are added to the culture media to increase production of proteins in cells. These inducers induce the cell to produce more desired product. One such agent is sodium butyrate. However, the drawback of using sodium butyrate in cell culture is that it affects significantly cell viability. For instance Kim et al (2004) have shown that although sodium butyrate was able to increase protein production in recombinant CHO cells in a batch culture, at the end of the production run (after 8 days of culture), cell viability was less than 45%. Repeating the same experiments in perfusion batch culture, the authors noticed that within 6 days of treatment, cell viability was as low as 15%, whereas a typical production period can be up to 40-45 days in perfusion mode.

Because many proteins are recombinantly produced in perfusion mode by cells grown in culture for more up to 40-45 days, there is a need for methods allowing for more efficient production runs owing to increased viable cell density, for a given perfusion rate, and/or maintenance of an acceptable cell viability over a long time, leading to an increased titre.

Therefore, there remains a need for culture conditions and production methods allowing for more efficient production runs owing to increased viable cell density, for a given perfusion rate, and/or maintenance of an acceptable cell viability over a long time, leading to an increased tire. The present invention addresses this need by providing methods and compositions allowing for more efficient production runs.

### SUMMARY OF THE INVENTION

In one aspect the invention provides a method of producing a recombinant protein in perfusion mode, said method comprising culturing a mammalian host cell expressing said recombinant protein in a concentrated cell culture medium.

In a further aspect, the invention provides a method of culturing in perfusion mode a mammalian host cell that expresses a recombinant protein, said method comprising culturing said host cell in a concentrated cell culture medium.

In another aspect, the invention provides a method of increasing production in perfusion mode of a recombinant protein, said method comprising culturing a mammalian host cell expressing said protein in a concentrated cell culture medium.

In a further aspect, the invention provides a concentrated cell culture medium, wherein said concentrated cell culture medium is to be used in perfusion method.

In yet another aspect, herein is provided a method for producing a concentrated cell culture medium, comprising a) mixing all together the main components at the desired concentration, between 1.5× to 5× the concentration of a standard medium not concentrated to provide a primary concentrated cell culture medium, b) optionally adding to the primary concentrated cell culture medium of step a) the remaining components which cannot be concentrated without altering the quality of the medium and/or those needed to adjust specific characteristics of the medium and c) adding the salt in order to adjust the osmolality.

In any of the method including the concentrated cell culture medium or in any of the concentrated cell culture medium herein described, said concentrated cell culture medium can be further supplemented with at least one salt in order to adjust the osmolality of said medium.

### BRIEF DESCRIPTION OF THE FIGURES

FIG. 1 shows an exemplary concentrate medium without affecting compound balance and keeping the osmolality constant

FIG. 2 shows VCD profiles of cells mAb1 of batch culture duplicates run with the following media: 01-02 PM: Fed-batch production media (control)/03-04 PM 1×: Fed-batch platform media mimic using depleted powder/05-06 PM 1.5×: Media enriched by a factor 1.5/07-08 PM 2×: Media enriched by a factor 2. A) From day 0 to day 6, B) same experiences continued up to day 10.

FIG. 3 shows Viability profiles of cells mAb1 of batch culture duplicates run with the following media: 01-02 PM: Fed-batch production media (control)/03-04 PM 1×: Fed-batch platform media mimic using depleted powder/05-06 PM 1.5×: Media enriched by a factor 1.5/07-08 PM 2×: Media enriched by a factor 2. A) From day 0 to day 6, B) same experiences continued up to day 10.

FIG. 4 shows VCD profiles of cells mAb2 of batch culture duplicates run with the following media: 01-02 PM: Fed-batch production media (control)/03-04 PM 1×: Fed-batch platform media mimic using depleted powder/05-06 PM 1.5×: Media enriched by a factor 1.5/07-08 PM 2×: Media enriched by a factor 2. A) From day 0 to day 6, B) same experiences continued up to day 10.

FIG. 5 shows Viability profiles of cells mAb2 of batch culture duplicates run with the following media: 01-02 PM: Fed-batch production media (control)/03-04 PM 1×: Fed-batch platform media mimic using depleted powder/05-06 PM 1.5×: Media enriched by a factor 1.5/07-08 PM 2×: Media enriched by a factor 2. A) From day 0 to day 6, B) same experiences continued up to day 10.

FIG. 6 shows titre (Biacore®) in relation to time (from day 6 to day 10) for the host cells expressing the antibody mAb1. Batch culture duplicates were run with the following media: 01-02 PM: Fed-batch production media (control)/03-04 PM 1×: Fed-batch platform media mimic using depleted powder/05-06 PM 1.5×: Media enriched by a factor 1.5/07-08 PM 2×: Media enriched by a factor 2.

FIG. 7 shows titre (Biacore®) in relation to time (from day 6 to day 10) for the host cells expressing the antibody mAb2. Batch culture duplicates were run with the following media: 01-02 PM: Fed-batch production media (control)/03-04 PM 1×: Fed-batch platform media mimic using depleted powder/05-06 PM 1.5×: Media enriched by a factor 1.5/07-08 PM 2×: Media enriched by a factor 2.

FIG. 8 shows VCD profiles of cells FP of batch culture duplicates run with the following media: 01-02 PM: Fed-batch production media (control)/03-04 PM 1×: Fed-batch platform media mimic using depleted powder/05-06 PM 1.5×: Media enriched by a factor 1.5/07-08 PM 2×: Media enriched by a factor 2.

FIG. 9 shows Viability profiles of cells FP of batch culture duplicates run with the following media: 01-02 PM: Fed-batch production media (control)/03-04 PM 1×: Fed-batch platform media mimic using depleted powder/05-06 PM 1.5×: Media enriched by a factor 1.5/07-08 PM 2×: Media enriched by a factor 2.

FIG. 10 shows titre (Biacore®) in relation to time (from day 7 to day 10) for the host cells expressing the fusion protein FP. Batch culture duplicates were run with the following media: 01-02 PM: Fed-batch production media (control)/03-04 PM 1×: Fed-batch platform media mimic using depleted powder/05-06 PM 1.5×: Media enriched by a factor 1.5/07-08 PM 2×: Media enriched by a factor 2.

### DETAILED DESCRIPTION OF THE INVENTION

In the case of conflict, the present specification, including definitions, will control.

Unless defined otherwise, all technical and scientific terms used herein have the same meaning as is commonly understood by one of skill in art to which the subject matter herein belongs. As used herein, the following definitions are supplied in order to facilitate the understanding of the present invention.

As used in the specification and claims, the term “and/or” used in a phrase such as “A and/or B” herein is intended to include “A and B”, “A or B”, “A”, and “B”.

As used in the specification and claims, the term “cell culture” or “culture” is meant the growth and propagation of cells in vitro, i.e. outside of an organism or tissue. Suitable culture conditions for mammalian cells are known in the art, such as taught in Cell Culture Technology for Pharmaceutical and Cell-Based Therapies (2005). Mammalian cells may be cultured in suspension or while attached to a solid substrate.

The terms “cell culture medium,” “culture medium”, “medium,” and any plural thereof, refer to any medium in which cells of any type can be cultured. A “basal medium” refers to a cell culture medium that contains all of the essential ingredients useful for cell metabolism. This includes for instance amino acids, lipids, carbon source, vitamins and mineral salts. DMEM (Dulbeccos' Modified Eagles Medium), RPMI (Roswell Park Memorial Institute Medium) or medium F12 (Ham's F12 medium) are examples of commercially available basal media. Alternatively, said basal medium can be a proprietary medium fully developed in-house, also herein called “chemically defined medium” or “chemically defined culture medium”, in which all of the components can be described in terms of the chemical formulas and are present in known concentrations. The culture medium can be free of proteins and/or free of serum, and can be supplemented by any additional compound(s) such as amino acids, salts, sugars, vitamins, hormones, growth factors, depending on the needs of the cells in culture. Said basal medium will herein alternatively be called production medium or production culture medium. In the context of the present invention, when the terms “cell culture medium,” “culture medium” or “medium” are used in relation with perfusion method, said medium can alternatively be called “perfusion medium”. The same applies to “basal medium” that could alternatively be called “perfusion basal medium”.

The term “bioreactor” or “culture system” refers to any system in which cells can be cultured. This term includes but is not limited to flasks, static flasks, spinner flasks, tubes, shake tubes, shake bottles, wave bags, bioreactors, fibre bioreactors, fluidised bed bioreactors, and stirred-tank bioreactors with or without microcarriers. Alternatively, the term “culture system” also includes microtitre plates, capillaries or multi-well plates. Any size of bioreactor can be used, for instance from 0.1 millilitre (0.1 mL, very small scale) to 20000 litres (20000 L or 20 KL, large scale), such as 0.1 mL, 0.5 mL 1 mL, 5 mL, 0.01 L, 0.1 L, 1 L, 2 L, 5 L, 10 L, 50 L, 100 L, 500 L, 1000 L (or 1 KL), 2000 L (or 2 KL), 5000 L (or 5 KL), 10000 L (or 10 KL), 15000 L (or 15 KL) or 20000 L (20 KL). In perfusion mode, bioreactors of between 1 L to 2 KL are usually used.

The term “perfusion” refers to a method of growing cells in which the cell culture receives fresh perfusion medium, or fresh perfusion basal medium, while simultaneously removing spent medium. Perfusion can be continuous, step-wise, intermittent, or a combination of any or all of any of these. Perfusion rates can be less than a working volume to many working volumes per day. Preferably the cells are retained in the culture and the spent medium that is removed is substantially free of cells or has significantly fewer cells than the culture. Perfusion can be accomplished by a number of cell retention techniques including centrifugation, sedimentation, or filtration (see for example Voisard et al., 2003). When using the methods and/or cell culture techniques of the instant invention, the recombinant protein are generally directly secreted into the culture medium. Once said protein is secreted into the medium, supernatants from such expression systems can be first concentrated using a commercially available protein concentration filter. As of today, it does not exist a way to perform perfusion at lab scale (in very small volume). However, it appears that the main trends observed in batch culture generally remain similar in perfusion culture (Continuous bioprocess: current practice and future potential, 2014). Usually, only fine tuning is needed. Therefore small bioreactors can be used to screen a high numbers of media or of conditions to be then applied to perfusion cell culture.

As used herein, “cell density” refers to the number of cells in a given volume of culture medium. “Viable cell density” (or VCD) refers to the number of live cells in a given volume of culture medium, as determined by standard viability assays. The terms “Higher cell density” or “Higher viable cell density”, and equivalents thereof, means that the cell density or viable cell density is increased by at least 15% when compared to the control culture condition, for a given perfusion rate. The cell density will be considered as maintained if it is in the range of −15% to 15% compared to the control culture condition, for a given perfusion rate. The terms “Lower cell density” or “Lower viable cell density”, and equivalents thereof, means that the cell density or viable cell density is decreased by at least 15% when compared to the control culture condition, for a given perfusion rate.

The term “viability”, or “cell viability” refers to the ratio between the total number of viable cells and the total number of cells in culture. Viability is usually acceptable as long as it is at not lower than 60%. Viability is often used to determine time for harvest.

The wording “titre” refers to the amount or concentration of a substance, here the protein of interest, in solution. It is an indication of the number of times the solution can be diluted and still contain detectable amounts of the molecule of interest. It is calculated routinely for instance by diluting serially (1:2, 1:4, 1:8, 1:16, etc) the sample containing the protein of interest and then using appropriate detection method (colorimetric, chromatographic etc.), each dilution is assayed for the presence of detectable levels of the protein of interest. Titre can also be measured by means such as by fortéBIO Octet® or with Biacore C®, as used in the example section. In perfusion mode, it is usually referred to the harvest titre. Titre can also be measured in the bioreactor, before harvest.

The term “specific productivity” refers to the amount of a substance, here the protein of interest, produced per cell per day.

The terms “higher titre” or “higher productivity”, and equivalents thereof, means that the titre or the productivity is increased by at least 10% when compared to the control culture condition. The titre or specific productivity will be considered as maintained if it is in the range of −10% to 10% compared to the control culture condition. The terms “lower titre” or “lower productivity”, and equivalents thereof, means that the titre or the productivity is decreased by at least 10% when compared to the control culture condition.

The term “protein” as used herein includes peptides and polypeptides and refers to compound comprising two or more amino acid residues. A protein according to the present invention includes but is not limited to a cytokine, a growth factor, a hormone, a fusion protein, an antibody or a fragment thereof. A therapeutic protein refers to a protein that can be used or that is used in therapy.

The term “recombinant protein” means a protein produced by recombinant technics. Recombinant technics are well within the knowledge of the skilled person (see for instance Sambrook et al., 1989, and updates).

As used in the specification and claims, the term “antibody”, and its plural form “antibodies”, includes, inter alia, polyclonal antibodies, affinity-purified polyclonal antibodies, monoclonal antibodies, and antigen-binding fragments, such as F(ab′)2, Fab proteolytic fragments, and single chain variable region fragments (scFvs). Genetically engineered intact antibodies or fragments, such as chimeric antibodies, scFv and Fab fragments, as well as synthetic antigen-binding peptides and polypeptides, are also included.

The term “humanised” immunoglobulin refers to an immunoglobulin comprising a human framework region and one or more CDRs from a non-human (usually a mouse or rat) immunoglobulin. The nonhuman immunoglobulin providing the CDRs is called the “donor” and the human immunoglobulin providing the framework is called the “acceptor” (humanisation by grafting non-human CDRs onto human framework and constant regions, or by incorporating the entire non-human variable domains onto human constant regions (chimerisation). Constant regions need not be present, but if they are, they must be substantially identical to human immunoglobulin constant regions, i.e., at least about 85-90%, preferably about 95% or more identical. Hence, all parts of a humanised immunoglobulin, except possibly the CDRs and a few residues in the heavy chain constant region if modulation of the effector functions is needed, are substantially identical to corresponding parts of natural human immunoglobulin sequences. Through humanising antibodies, biological half-life may be increased, and the potential for adverse immune reactions upon administration to humans is reduced.

As used in the specification and claims, the term “fully human” immunoglobulin refers to an immunoglobulin comprising both a human framework region and human CDRs. Constant regions need not be present, but if they are, they must be substantially identical to human immunoglobulin constant regions, i.e., at least about 85-90%, preferably about 95% or more identical. Hence, all parts of a fully human immunoglobulin, except possibly few residues in the heavy chain constant region if modulation of the effector functions or pharmacokinetic properties are needed, are substantially identical to corresponding parts of natural human immunoglobulin sequences. In some instances, amino acid mutations may be introduced within the CDRs, the framework regions or the constant region, in order to improve the binding affinity and/or to reduce the immunogenicity and/or to improve the biochemical/biophysical properties of the antibody.

The term “recombinant antibodies” means antibodies produced by recombinant technics. Because of the relevance of recombinant DNA techniques in the generation of antibodies, one needs not be confined to the sequences of amino acids found in natural antibodies; antibodies can be redesigned to obtain desired characteristics. The possible variations are many and range from the changing of just one or a few amino acids to the complete redesign of, for example, the variable domain or constant region. Changes in the constant region will, in general, be made in order to improve, reduce or alter characteristics, such as complement fixation (e.g. complement dependent cytotoxicity, CDC), interaction with Fc receptors, and other effector functions (e.g. antibody dependent cellular cytotoxicity, ADCC), pharmacokinetic properties (e.g. binding to the neonatal Fc receptor; FcRn). Changes in the variable domain will be made in order to improve the antigen binding characteristics. In addition to antibodies, immunoglobulins may exist in a variety of other forms including, for example, single-chain or Fv, Fab, and (Fab′)2, as well as diabodies, linear antibodies, multivalent or multispecific hybrid antibodies.

As used herein, the term “antibody portion” refers to a fragment of an intact or a full-length chain or antibody, usually the binding or variable region. Said portions, or fragments, should maintain at least one activity of the intact chain/antibody, i.e. they are “functional portions” or “functional fragments”. Should they maintain at least one activity, they preferably maintain the target binding property. Examples of antibody portions (or antibody fragments) include, but are not limited to, “single-chain Fv”, “single-chain antibodies,” “Fv” or “scFv”. These terms refer to antibody fragments that comprise the variable domains from both the heavy and light chains, but lack the constant regions, all within a single polypeptide chain. Generally, a single-chain antibody further comprises a polypeptide linker between the VH and VL domains which enables it to form the desired structure that would allow for antigen binding. In specific embodiments, single-chain antibodies can also be bi-specific and/or humanised.

A “Fab fragment” is comprised of one light chain and the variable and CH1 domains of one heavy chain. The heavy chain of a Fab molecule cannot form a disulfide bond with another heavy chain molecule. A “Fab′ fragment” that contains one light chain and one heavy chain and contains more of the constant region, between the CH1 and CH2 domains, such that an interchain disulfide bond can be formed between two heavy chains is called a F(ab′)2 molecule. A “F(ab′)2” contains two light chains and two heavy chains containing a portion of the constant region between the CH1 and CH2 domains, such that an interchain disulfide bond is formed between two heavy chains. Having defined some important terms, it is now possible to focus the attention on particular embodiments of the instant invention.

Examples of known antibodies which can be produced according to the present invention include, but are not limited to, adalimumab, alemtuzumab, belimumab, bevacizumab, canakinumab, certolizumab, pegol, cetuximab, denosumab, eculizumab, golimumab, infliximab, natalizumab, ofatumumab, omalizumab, pertuzumab, ranibizumab, rituximab, siltuximab, tocilizumab, trastuzumab, ustekinumab or vedolizomab.

The terms “Inducing agent”, “inducer” or “productivity enhancer” refer to a compound or a composition (such a culture medium) allowing an increase of the production performance or of the protein production when added in cell cultures. For instance, one of the inducers known for E. coli production is IPTG (Isopropyl β-D-1-thiogalactopyranoside) and inducers for CHO production are among others sodium butyrate, doxycycline or dexamethasone.

The term “subject” is intended to include (but not limited to) mammals such as humans, dogs, cows, horses, sheep, goats, cats, mice, rabbits, or rats. More preferably, the subject is a human.

Key aspects of perfusion culture are the media composition and the perfusion rate. The perfusion rate will determine the amount of nutrients available for the cells, and the media composition the ratio between these different compounds. The proper balance of cell culture media is essential for the cell metabolism. Any excess of compound could lead to an unwanted pathway and accumulation of some sort of toxic compound that could harm the culture viability. Once a well-balanced media is available, the perfusion rate can be modified to adjust the nutrient addition to the cell's needs. Increasing the perfusion rate will bring more nutrients to the cells. By-products are of course secreted by the cells and need to be eliminated. Again, the perfusion rate can be modified to tune this elimination capacity. The waste compound removal rate is increased when the perfusion rate is increased.

Different approaches can be taken to optimize a perfusion culture. If the goal is to minimize the perfusion rate to limit the flowrates. In order to decrease the perfusion rate without changing the compound addition rates, one needs to enrich the media. The present invention describes a method to enrich an already existing cell culture media without affecting the ratio between the main compounds and keeping the osmolality constant.

The present invention provides methods and media for allowing for more efficient production runs in perfusion mode (perfusion cell culture), owing to increased viable cell density, for a given perfusion rate, and/or maintenance of an acceptable cell viability over a longer time, for a given perfusion rate, leading to an increased titre of the protein to be produced and decreased media volume consumption. The present invention is based on the optimisation of cell culture media for protein manufacturing, such as production of antibodies or antigen-binding fragments, resulting in more efficient production runs, increase of viable cell density for a given perfusion rate, maintenance of viability and increase of the total production of a recombinant protein for an equivalent media volume consumption.

The inventors have surprisingly found that when mammalian cells are cultivated in perfusion mode in a concentrated cell culture medium, the viable cell density for a given perfusion rate is increased, and substantial or significant decrease in cell viability over the production period is avoided (i.e. more efficient production runs), when compared to the same mammalian cells cultivated in perfusion mode (same perfusion rate, same ratios between the main components, same osmolality) in a cell culture medium not concentrated. Further, the total production of a recombinant protein can be increased (i.e. the titre can be increased). Thus, when it is desirable to increase efficiency of production runs, in perfusion mode, a concentrated cell culture medium can be used.

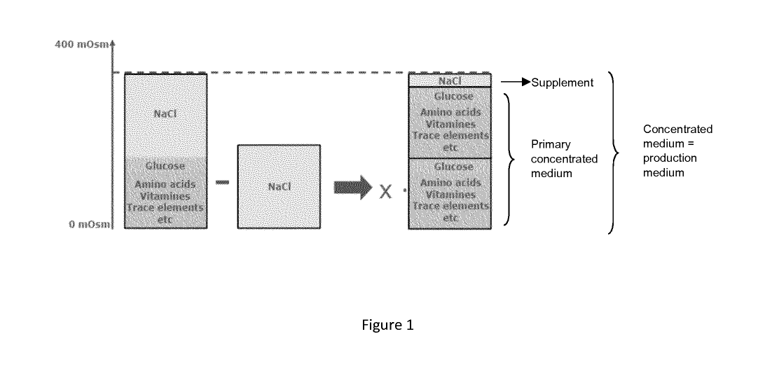

In one aspect, the invention provides a concentrated cell culture medium, wherein the components, at least the main components, of said concentrated cell culture medium are at concentrations (alternatively are present in amounts) of or of about 1.5 to 5 fold (1.5× to 5×) higher than the ones of a similar culture medium not concentrated (i.e. comprising the same main components in the same ratios). Preferably, said components of said concentrated cell culture medium are at concentrations of or of about 1.5×, 2×, 2.5×, 3×, 3.5×, 4×, 4.5× or 5× fold (1.5× to (5×) higher than the ones of a similar culture medium not concentrated. Even more preferably, said components of said concentrated cell culture medium are at concentrations of or of about 1.5×, 2×, 2.5×, 3×, higher than the ones of a similar culture medium not concentrated. As examples, if in a culture medium not concentrated, cysteine is at 10 mg/L and Glutamine at 2 mg/L (ratio cysteine/glutamine is 5:1), then a 1.5× concentrated medium will contain 15 mg/L of cysteine and 3 mg/L glutamine, and a 5× concentrated medium will contain 50 mg/L of cysteine and 10 mg/L glutamine. In both case the ratio cysteine/glutamine will stay at 5:1. The same principle will be automatically applied to all the main components of the concentrated culture medium. The concentrated culture medium that is to be used as a production medium is composed of two elements: a primary concentrated medium, usually formulated as a powder, and at least one supplement (see FIG. 1). Indeed, some of the components of a medium cannot be concentrated without impacting the quality of the medium (e.g. aggregation, solubility, etc). Some of the components are also used to adjust specific characteristics of the medium (eg. osmolality, pH, etc). Therefore such components will not be part of the primary concentrated medium, but will be added as supplement(s). This or these remaining component(s) will be added, as supplement(s), to the primary concentrated medium after its formulation, once in the liquid form for instance if the primary concentrated medium is formulated as a powder. This is the case for instance for the salt that can provide up to half of the osmolality of a culture medium. Said salt can be NaCl for instance. In the context of the present invention, the primary concentrated cell culture medium is first formulated at least without salt, either as a liquid or as a powder. Said salt will be added later on, after formulation of the primary concentrated medium, once in the liquid form for instance if the primary concentrated medium is provided as a powder, in order to adjust the osmolality. Preferably the osmolality of the concentrated culture medium will be the same as the one of the comparable cell culture medium that is not concentrated. Alternatively, it can be added as a supplement directly in the cell culture. The same can be applied to the other possible components which cannot be concentrated without impacting the quality of the medium, and/or those needed to adjust specific characteristics of the medium. As the salt, they can be added later on, after formulation of the primary concentrated medium, once in the liquid form for instance if the primary concentrated medium is provided as a powder. Alternatively, they can be added as a supplement directly in the cell culture.

For the purposes of this invention, a cell culture medium (concentrated or not) is a medium suitable for growth of mammalian cells in in vitro cell culture in perfusion mode. Said medium is preferably a chemically defined medium. Preferably, the cell culture medium is free of animal components; they can be serum-free and/or protein-free.

Also, in the context of this invention, it is important to respect the final properties (or characteristics) of the liquid media such as osmolality and pH. Osmolality can be adjusted using the salt that was removed from the main powder formulation and pH with acid or base depending on needs. The pH can be adjusted using a correct amount of buffer salt, which contribution to osmolality of course needs to be taken into account.

Also described herein is a primary concentrated cell culture medium for use in cell culture in perfusion mode, wherein said primary concentrated cell culture medium at the time of its formulation is preferably depleted at least in salt. Said primary culture medium can be formulated as a powder or in liquid form.

In another aspect, the invention provides a method for producing a concentrated cell culture medium, comprising a) mixing all together the main components at the desired concentration between 1.5× to 5× the concentration of a standard medium not concentrated to provide a primary concentrated cell culture medium, b) optionally adding to the primary concentrated cell culture medium of step a) the remaining components which cannot be concentrated without altering the quality of the medium and/or those needed to adjust specific characteristics of the medium and c) adding the salt in order to adjust the osmolality.

In a further aspect the invention provides use of the concentrated cell culture medium according to the present invention in a perfusion culture as an inducer in a cell culture.

In another aspect the invention provides a method of producing a recombinant protein in perfusion mode, said method comprising culturing a mammalian host cell expressing said recombinant protein in one of the concentrated cell culture medium according to the invention. In some preferred embodiments, the host cell is Chinese Hamster Ovary (CHO) cells.

Alternatively, the invention provides a method of culturing in perfusion mode a mammalian host cell that expresses a recombinant protein, said method comprising culturing said host cell in one of the concentrated cell culture medium according to the present invention. In some preferred embodiments, the host cell is Chinese Hamster Ovary (CHO) cells.

In a further aspect the invention provides a method of increasing production in perfusion mode of a recombinant protein, said method comprising culturing a mammalian host cell expressing said protein in one of the concentrated cell culture medium according to the present invention. In some preferred embodiments, the host cell is Chinese Hamster Ovary (CHO) cells.

When a concentrated cell culture medium according to the invention as a whole is used, a higher cell density can be supported for a similar perfusion rate. Cell viability does not substantially or significantly decrease and total production of the recombinant protein can be increased relative to cells grown a similar medium wherein the components are not concentrated.

As used herein, the phrase “cell viability does not substantially or significantly decrease”, when compared to cells grown without the concentrated medium, means that cell viability does not decrease more than about 15% compared to control cultures (i.e. cells grown without a concentrated medium).

In an embodiment of the present invention, the mammalian host cell (herein also refer to as a mammalian cell) is preferably selected from the group of, but not limited to, HeLa, Cos, 3T3, myeloma cell lines (for instance NS0, SP2/0), and Chinese hamster ovary (CHO) cells. In a preferred embodiment, the host cell is Chinese Hamster Ovary (CHO) cells.

In the context of the invention as a whole, the recombinant mammalian cell, is grown in a culture system such as a bioreactor. The bioreactor is inoculated with viable cells in a culture medium. Said culture medium is preferably the concentrated culture medium according to the invention. Alternatively, the bioreactor is inoculated with viable cells in a culture medium at 1× concentration and at any time after the start of the culture a concentrated medium is used instead of the 1× culture medium. Preferably the culture medium is serum free and/or protein-free. Once inoculated into the production bioreactor the recombinant cells undergo an exponential growth phase. The perfusion mode can be activated anytime it is required to support the cell growth and should ideally stay constant during the run. Once the desired cell density is reached a bleed can be activated in order to maintain this cell density constant. The media is therefore continuously fed at a flow rate that corresponds to the sum of harvest and bleed. The harvest is collected through a cell retention device such as an ATF™ for example.

The methods, media and uses according to the present invention may be used to improve the production runs and/or the total production of recombinant proteins, in perfusion mode, with one step or multistep (or multiple stages) culture processes. In a multiple stages process, cells are cultured in two or more distinct phases. For example cells are cultured first in one or more growth phases, under conditions improving cell proliferation and viability, then transferred to production phase(s), under conditions improving protein production. In a multistep culture process, some conditions may change from one step (or one phase) to the other: media composition, shift of pH, shift of temperature, etc. The growth phase can be performed at a temperature higher than in production phase. For example, the growth phase can be performed at a first temperature from about 35° C. to about 38° C., and then the temperature is shifted for the production phase to a second temperature from about 29° C. to about 37° C. The cell cultures can be maintained in production phase for weeks, as long as the process supports a good viability The mammalian cell lines (also referred to as “recombinant cells” or “host cells”) used in the invention are genetically engineered to express a protein of commercial or scientific interest.

Methods and vectors for genetically engineering of cells and/or cell lines to express a polypeptide of interest are well known to those of skill in the art; for example, various techniques are illustrated in Ausubel et al. (1988, and updates) or Sambrook et al. (1989, and updates). The methods or media of the invention can be used to culture cells that express recombinant proteins of interest. The recombinant proteins are usually secreted into the culture medium from which they can be recovered. The recovered proteins can then be purified, or partially purified using known processes and products available from commercial vendors. The purified proteins can then be formulated as pharmaceutical compositions. Suitable formulations for pharmaceutical compositions include those described in Remington's Pharmaceutical Sciences, 1995.

In the context of the invention as a whole, the recombinant protein is selected from the group consisting of an antibody or antigen binding fragment thereof, such as a human antibody or antigen-binding portion thereof, a humanised antibody or antigen-binding portion thereof, a chimeric antibody or antigen-binding portion thereof, a recombinant fusion protein, a growth factor, a hormone, or a cytokine.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

The foregoing description will be more fully understood with reference to the following examples. Such Examples, are, however, exemplary of methods of practising the present invention and are not intended to limit the scope of the invention.

### EXAMPLES

### Material and Methods

### I. Cells, Cell Expansion and Cell Growth

### 1) Cells

Assays were performed with 2 CHO cell lines:
CHO-S cells expressing IgG1 mAb1, herein “Cells mAb1” or “mAb1 cells”. “mAb1” is a fully human monoclonal antibody directed against a soluble protein. Its isoelectric point (pl) is about 8.20-8.30.
CHO-K1 cells expressing IgG1 mAb2, herein “Cells mAb2” or “mAb2 cells”. “mAb2” is a humanised monoclonal antibody directed against a receptor found on cell membranes. Its isoelectric point (pl) is about 9.30.
CHO-S cells expressing a fusion protein FP, herein “Cells FP” or “FP cells”. “FP” is an IgG1 fusion protein, comprising one part directed against a membrane protein (IgG part) linked to a second part targeting a soluble immune protein. Its isoelectric point (pl) is about 6.3-7.0

### 2) Cell Expansion

Cell expansion was performed in tubes in a medium suitable for cell expansion. Assays in perfusion started after at least one week expansion.

### 3) Inoculation

Cells expressing mAb1, mAb2 and FP were inoculated at 0.3×10⁶ cells per mL.

### 4) Batch and Perfusion

All assays were performed in batch culture, for mimicking perfusion process. The host cells were cultured in Spin Tubes® (working volume: 30 mL), and incubated at 36.5° C., 90% relative humidity, 5% CO2 and 320 rpm shaking for the experiments duration.

### II. Analytical Methods

Viable cell density (VCD) and viability are measured with the Guava easyCyte® flow cytometer or with the ViCell.

Antibody titres are measured either with Biacore C® or PA-HPLC depending on the molecule. Glycosylation profiles are established by capillary gel electrophoresis with laser-induced fluorescence (CGE-LIF). Dosages of aggregates and fragments were performed respectively via Size Exclusion High Performance Liquid Chromatography (SE-HPLC) and via SDS-capillary gel electrophoresis.

### Results and Discussions

### Example 1: Media Preparation

Medium 1 (not concentrated culture medium; herein “PM”) is prepared using:
Powder 1: containing main nutrients and salts (protein and/or serum free), all at a concentration 1×, and at a given osmolality “0”
Optionally a protein (should a protein containing medium be used)
Buffer salt for pH control (pH “P”).

Medium “PM 1×” herein correspond to “PM” but depleted in salt, except for pH control.

Medium 2 was prepared in a similar manner than PM in doubling the concentration of all the components of powder 1. However, doubling components concentration had a great impact on the osmolality of the solution. It was thus decided to formulate two media (hereafter PM 1.5× and PM 2×) based on a powder depleted at least in salt. This technic surprisingly allowed to concentrate the main nutrients without affecting the solution osmolality. Therefore, PM 1.5× and PM 2× were prepared as follow:
For PM 1.5×: Powder 3 (primary culture medium 3)=Powder 1 depleted with salts (optionally depleted in further compounds, e.g. compound Y), were the components are concentrated 1.5×; For PM 2×: Powder 4 (primary culture medium 4)=Powder 1 depleted with salts (optionally depleted in further compounds, e.g. compound Y), were the components are concentrated 2×
Optionally a protein (should PM 1.5× and/or PM 2× contain a protein)
Buffer salt for pH control (same pH “P” than for PM).
Salts to adjust osmolality
Optionally compound(s) Y if compound(s) Y was/were not included in the primary culture media 3 or 4.

It is important that the pH is adjusted to the desired level before completion of the media preparation. Once the correction needed is known, the buffer salt amount can be changed accordingly.

### Example 2: Impact of Media Concentration on Cell mAb1 and Cell mAb2

To evaluate the impact of the herein defined concentrated media on cell growth and metabolism, batch and perfusion cultures were run with control and different enrichment levels. All experiments were reproduced twice.

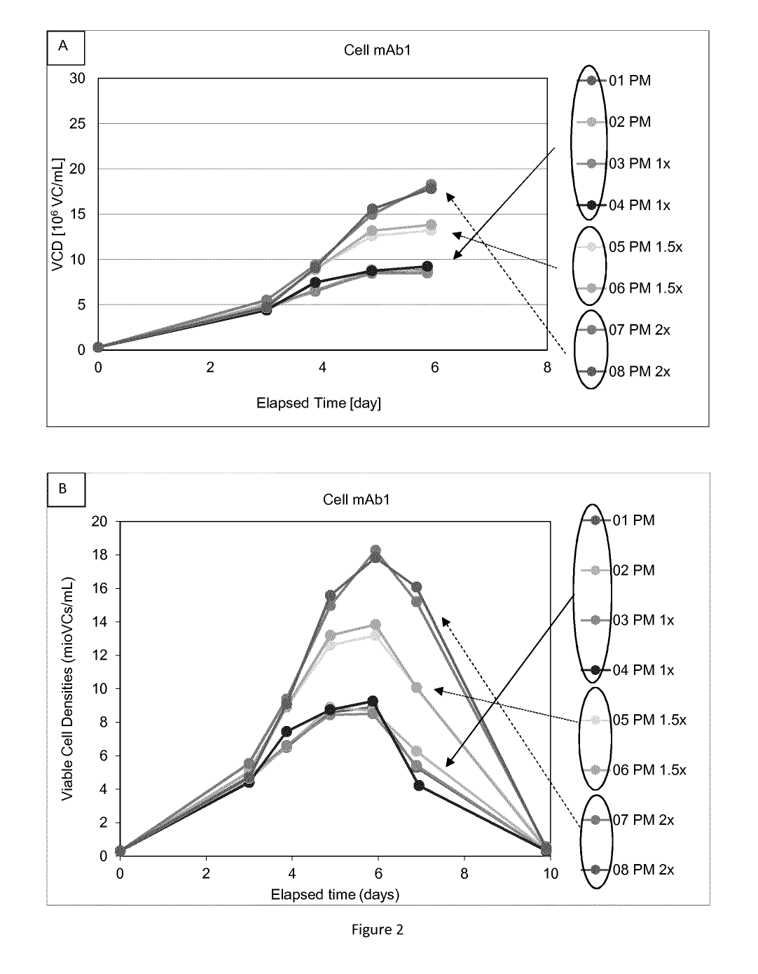

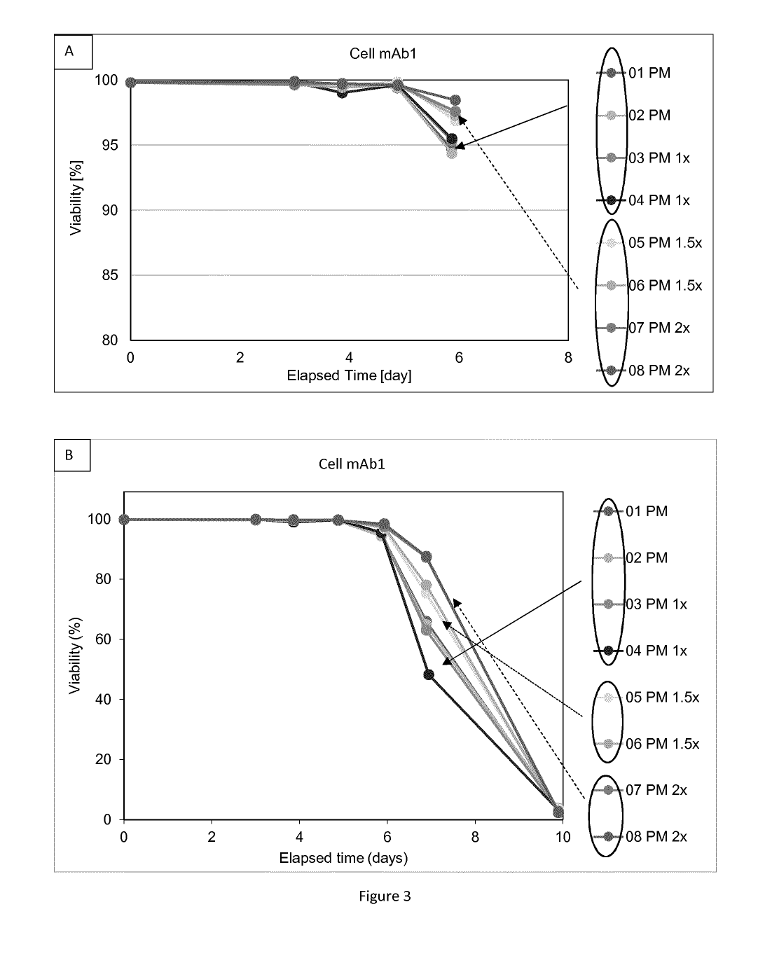

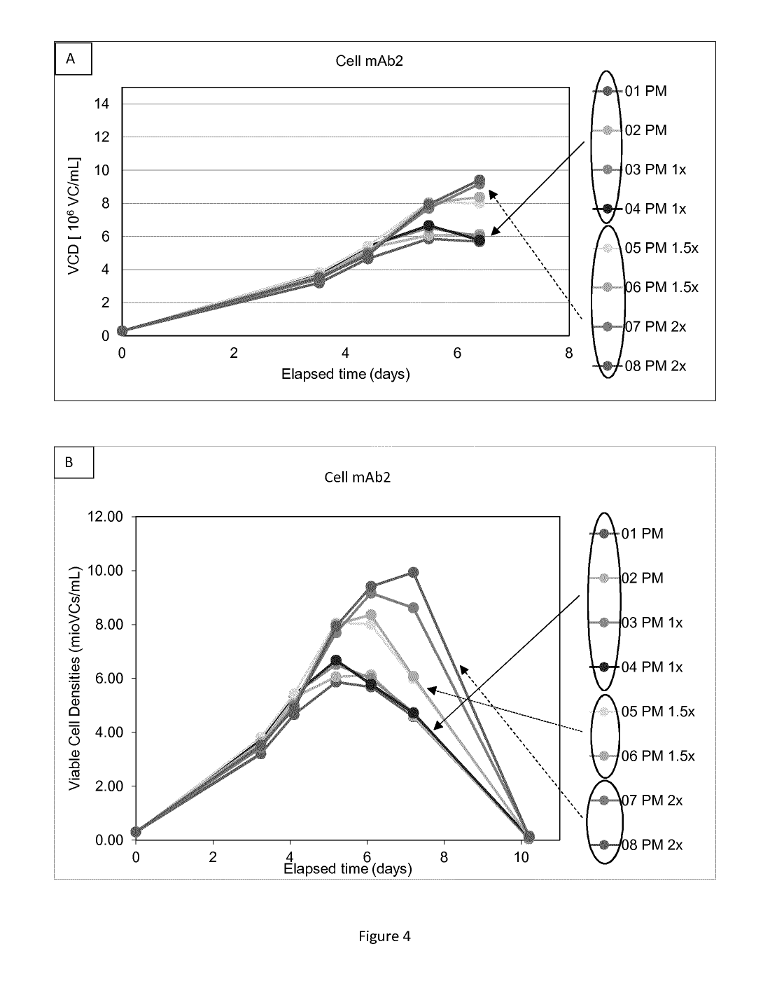

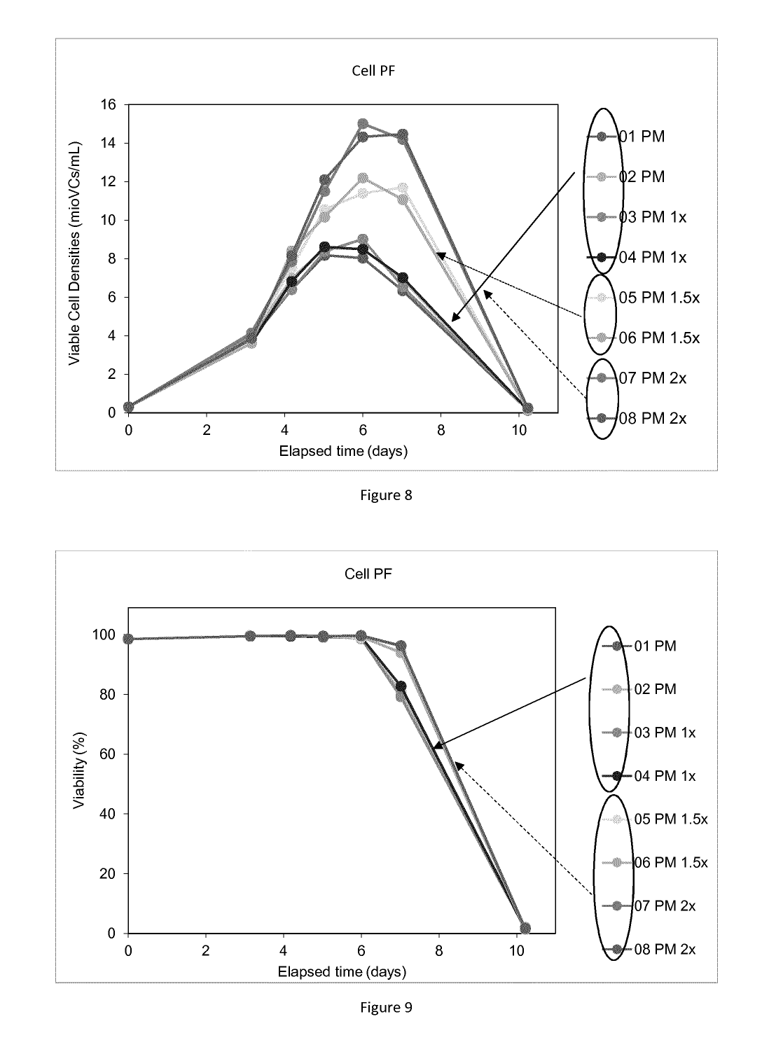

The results in batch cultures, for instance, revealed that:
Cells were able to grow from low cell densities (seeding density=0.3 million cells per mL) with media concentrated up to 2× (FIGS. 2, 4 and 8).
The media concentrated 1× (PM and PM1×) showed similar growth curves and viability profiles (FIGS. 2, 4 and 8).
The maximum VCD increased significantly (FIGS. 2, 4 and 8) with the concentrated media 1.5× (PM 1.5×) and 2× (PM 2×).
The concentrated media (PM 1.5× and PM 2×) delayed the viability drop (FIGS. 3, 5 and 9).

Conclusion: For all of the cells (cells mAb1, cells mAb2 and cells FP), the use of the present medium concentration strategy is promising as the performance in terms of VCD and viability were improved in preliminary runs.

### Example 3: Total Production of Proteins

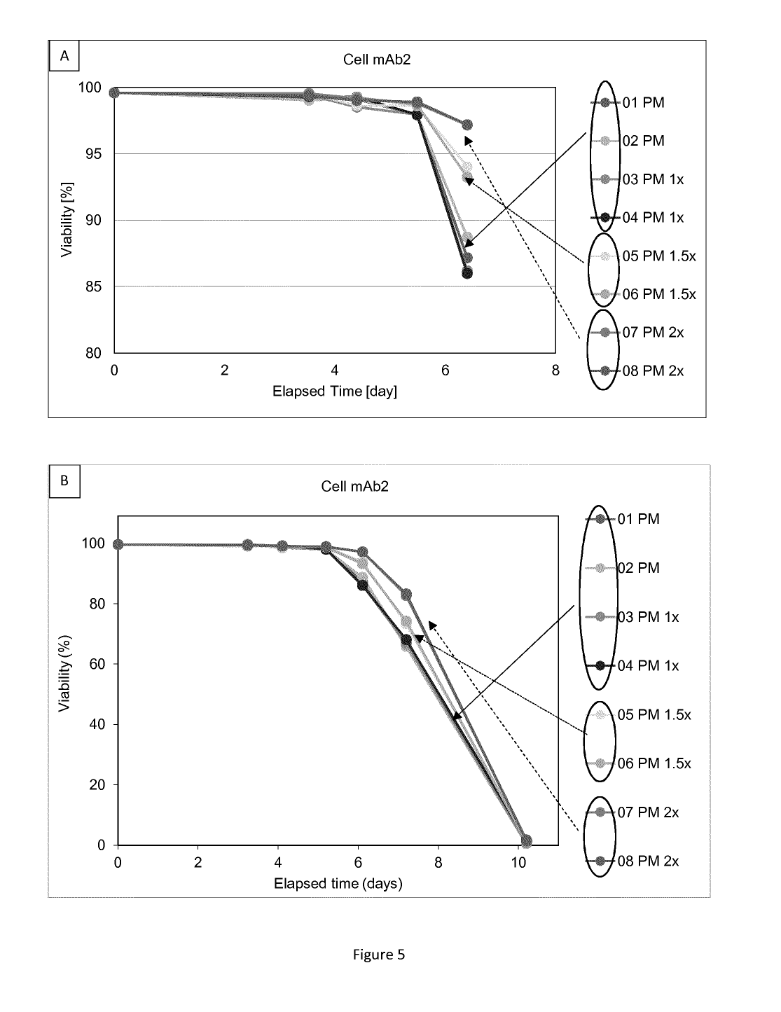

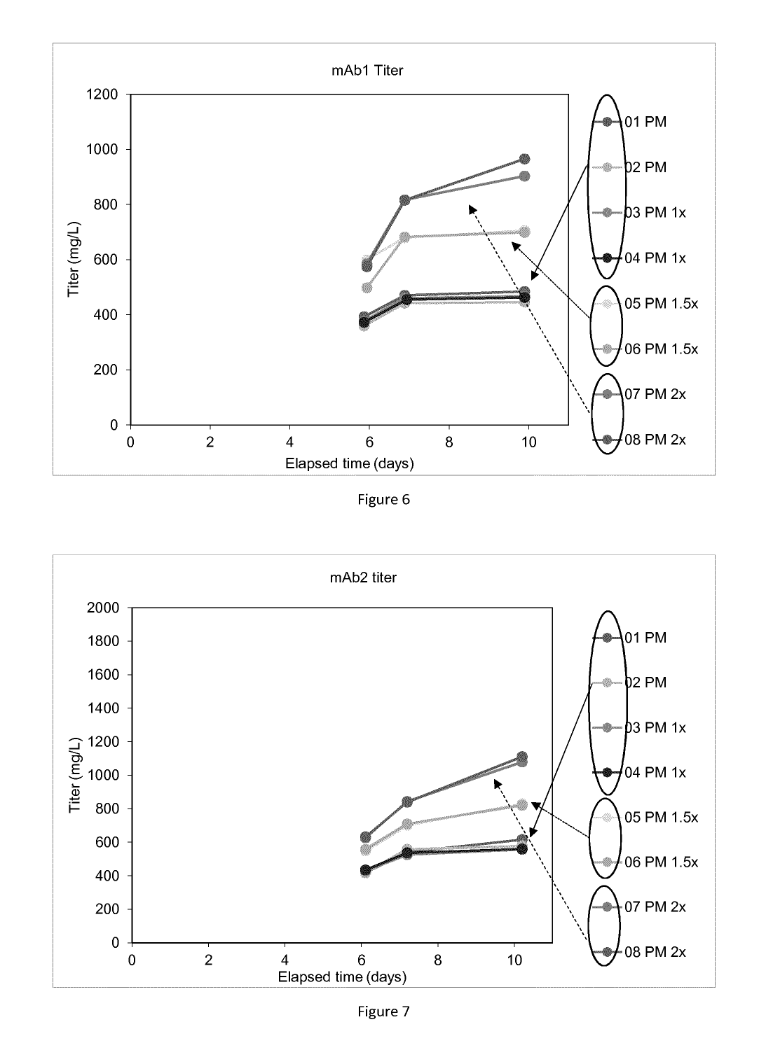

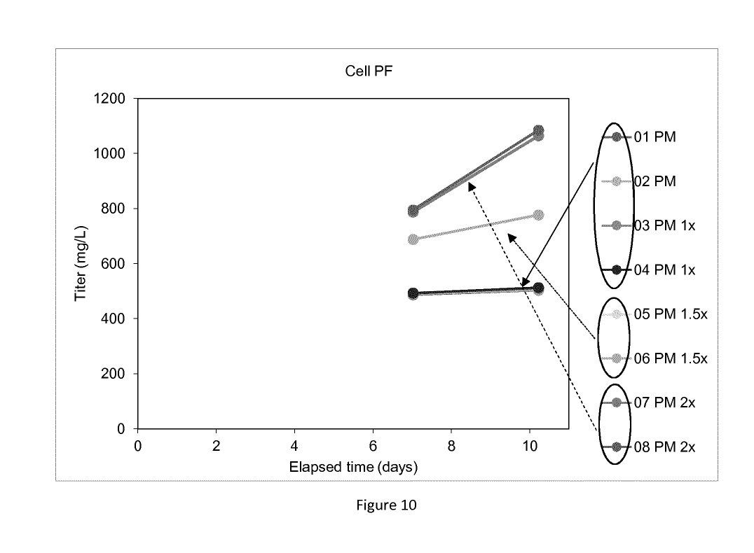

The efficacy of the concentrated medium strategy was also assessed vis-à-vis the titre of the antibodies mAb1 and mAb2, as well as the titre for the fusion protein FP. FIGS. 6, 7 and 10 underline that the titre of either antibodies mAb1 or mAb2 as well as of the fusion protein PF is greatly improved thanks to the concentrated medium strategy. The best improvements are observed in all cases with PM 2× (the titre is doubled or nearly doubled in each case). The improvements observed with PM 1.5× are already very interesting (the titre is increased by about 50% in each case). It is also anticipated that this strategy will not modify the glycosylation profile of the protein:

### Overall Conclusion

The use of concentrated media is a very promising strategy for enhancing the efficacy of the production runs and for increasing the quantity of the produced recombinant protein. Said concentrated media could thus be used as inducers.

The skilled person will understand from the results of the examples above that he can use concentrated media for modulating the efficacy of production runs and the production profile of any antibodies and any proteins, whatever the cell line that is used for production. The optimal concentration factor of the medium to be used in the culture will have to be determined case by case. This determination can be done without involving any inventive skill, based on the teaching of the present invention.

### REFERENCES

1) Kim et al., 2004, Biotechnol. Prog., 20:1788-1796
2) Continuous Bioprocessing: current practice & future potential. 2014. Ed. Refine technology. http://www.continuous-bioprocessing.com/
3) Voisard et al., 2003, Biotechnol. Bioeng. 82:751-765
4) Ausubel et al., 1988 and updates, Current Protocols in Molecular Biology, eds. Wiley & Sons, New York.
5) Sambrook et al., 1989 and updates, Molecular Cloning: A Laboratory Manual, Cold Spring Laboratory Press.
6) Remington's Pharmaceutical Sciences, 1995, 18th ed., Mack Publishing Company, Easton, Pa.

1-14. (canceled)

15. A method of producing a recombinant protein in perfusion mode, said method comprising culturing a mammalian host cell expressing said recombinant protein in a concentrated cell culture medium.

16. The method according to claim 15, wherein said method increases the efficiency of production runs.

17. The method according to claim 16, wherein the efficiency of production runs is measured by an increase of the viable cell density and/or a lower decrease in cell viability.

18. The method according to claim 15, wherein the host cell is selected from the group consisting of HeLa, Cos, 3T3, NS0, SP2/0, and a Chinese Hamster Ovary (CHO) cells.

19. The method according to claim 15, wherein the main components of the concentrated cell culture medium are present in amounts 1.5 to 5 times to the level of the amounts present in a comparable cell culture medium that is not concentrated.

20. The method according to claim 19, wherein the main components of the concentrated cell culture medium are present in amounts 1.5 to 3 times the level of the amounts present in a comparable cell culture medium that is not concentrated.

21. The method according to claim 15, wherein the recombinant protein is selected from the group consisting of a recombinant fusion protein, a growth factor, a hormone, a cytokine, an antibody or antigen binding fragment thereof, a humanized antibody or antigen-binding portion thereof, a chimeric antibody or antigen-binding portion thereof.

22. A method of culturing in perfusion mode a mammalian host cell that expresses a recombinant protein, said method comprising culturing said host cell in a concentrated cell culture medium.

23. A method of increasing production of a recombinant protein in perfusion mode, said method comprising culturing a mammalian host cell expressing said protein in a concentrated cell culture medium.

24. A concentrated cell culture medium, wherein said concentrated cell culture medium is to be used in cell culture in perfusion mode.

25. The concentrated cell culture medium according to claim 24, in which at least a salt is added after formulation of the primary concentrated cell culture medium in order to adjust the osmolality of said medium.

26. A primary concentrated cell culture medium for use in cell culture in perfusion mode, wherein said primary concentrated cell culture medium at the time of its formulation is depleted at least in salt.

27. The primary concentrated cell culture according to claim 26, in which at least a salt is added after formulation of the primary concentrated cell culture medium in order to adjust the osmolality of said medium.

28. A method for producing a concentrated cell culture medium, comprising a) mixing all together the main components at the desired concentration between 1.5× to 5× the concentration of a standard medium not concentrated to provide a primary concentrated cell culture medium, b) optionally adding to the primary concentrated cell culture medium of step a) the remaining components which cannot be concentrated without altering the quality of the medium and/or those needed to adjust specific characteristics of the medium and c) adding the salt in order to adjust the osmolality.
